(19) United States
(12) Patent Application Publication
(10) Pub. No.: US 2026/0295575 A1
(43) Pub. Date: Oct. 1, 2026

(54) CENTRIFUGE SYRINGE

(71) Applicant: HUBERTUS GOLLER GESELLSCHAFT M.B.H., Klosterneuburg (AT)

(72) Inventor: Michael Krapf-Günther, Klosterneuburg (AT)

(73) Assignee: HUBERTUS GOLLER GESELLSCHAFT M.B.H., Klosterneuburg (AT)

(21) Appl. No.: 19/479,582

(22) PCT Filed: Apr. 28, 2023
(86) PCT No.: PCT/EP2023/061278

§ 371 Date: Oct. 28, 2025

(51) Int. Cl. B01L 3/02; A61J 1/05; B04B 5/04; B04B 11/04

(52) U.S. Cl. CPC B01L 3/0217; B04B 5/0414; B04B 11/04; A61J 1/05; B01L 2200/0689; B01L 2300/041; B01L 2400/0409; B04B 2011/046

(57) ABSTRACT

The invention relates to a centrifuge syringe (1) comprising a syringe barrel (2) and a syringe plunger (3) axially displaceable in the syringe barrel (2), whereby a receiving volume (4) enclosed by the syringe barrel (2) and the syringe plunger (3) is formed for a body fluid, preferably blood (5), wherein the syringe plunger (3) has a plunger sealing section (8) and a plunger rod (9) undetachably formed on the plunger sealing section (8), wherein the syringe plunger (3) has a passage (15) extending axially through the plunger sealing section (8) and the plunger rod (9) for a fluid connection between the receiving volume (4) and a removal opening (16) at the end of the plunger rod (9) which faces away from the receiving volume (4), and wherein the syringe plunger (3) is of such a length that, if the syringe plunger (3) is in a position in the syringe barrel (2) with a minimum receiving volume (4), the removal opening (16) is arranged essentially at an end of the syringe barrel (2) opposite the receiving volume (4) or protrudes from the syringe barrel (2).

The invention relates to a centrifuge syringe comprising a syringe barrel and a syringe plunger axially displaceable in the syringe barrel, whereby a receiving volume enclosed by the syringe barrel and the syringe plunger is formed for a body fluid, preferably blood.

Centrifuge syringes are used to separate blood into its components: plasma, red blood cells and white blood cells. For this purpose, blood is drawn through a receiving opening in the syringe barrel into the receiving volume, which is delimited by the syringe plunger within the syringe barrel. Subsequently, the centrifuge syringe is inserted into a centrifuge so that the plasma as a lighter fraction is separated from the remaining components. However, if the plasma is present in the receiving volume in the area of the syringe plunger, technical challenges arise when extracting the plasma from the receiving volume. It is not possible in this case to use a conventional, one-piece syringe plunger, as it prevents the plasma from being removed.

In the prior art, for example, the document DE 2 147 823 is known for solving this problem. According to this document, the syringe plunger does not have a plunger rod formed integrally thereon, but has a comparatively short design in the axial direction of the syringe barrel so that the latter essentially consists of a plunger sealing section without a handle. Due to the low height, the syringe plunger can be provided with an axial passage so that a needle can be guided through this passage to withdraw blood plasma from the extraction opening. To manually displace the syringe plunger, a separate plunger rod must be clamped to the plunger.

Similar syringes are known from the documents DE 44 45 030 A1 , U.S. Pat. No. 8,052,969 B2, US20150209502A1, U.S. Pat. No. 9,050,403B2, U.S. Pat. No. 7,927,563B1 and WO 01/03756A1 . Therein, standard syringes or, respectively, removable plunger rods can be connected to the syringe plunger that is as small as possible, for example, via a Luer connection, so that plasma can be withdrawn from the receiving volume.

US20180333530A1 discloses a centrifuge syringe comprising a syringe barrel and a syringe plunger. The syringe plunger is also removable and must be removed during centrifugation. During centrifugation, the blood-filled syringe is oriented such that the lighter plasma fraction is located at the inlet/outlet opening of the syringe and can be forced out of the syringe barrel after the syringe plunger has been attached.

All those embodiments provide extremely cumbersome handling of the syringes, as their plungers can only be moved if a separate plunger rod or another syringe is temporarily connected to the plunger. If no plunger rod is connected to the plunger sealing section, the syringe cannot be operated. Moreover, US20180333530 involves the further disadvantage that mixing of the separated blood components is likely after centrifugation because the lower side of the syringe must face upwards until application.

The document DE 31 32 160 discloses a syringe having a plunger comprising a plunger sealing section and a plunger rod formed integrally thereon. After the plunger has been moved manually using the plunger rod, the latter can be broken off at a predetermined breaking point in order to allow access to the receiving volume. However, the plunger can no longer be moved afterwards. Thus, the disadvantages of the prior art as explained above also occur in this syringe.

The document DE 10 2007 056759 A1 likewise discloses a centrifuge syringe having a syringe barrel and a syringe plunger. The syringe plunger also has a plunger rod slidably supporting a plunger sealing section within the syringe. For withdrawing blood plasma after centrifugation, the blood plasma is to be guided out of the syringe through a receiving volume located next to the plunger rod.

U.S. Pat. No. 4,957,637 A discloses a centrifuge syringe which has a syringe barrel wherein, instead of a conventional syringe plunger, another syringe barrel is inserted into the first syringe barrel. By reducing the receiving volume, the plasma is merely transferred into the “plunger barrel” after centrifugation, rather than out of the port 24. To withdraw the plasma from the device, it must be “emptied” from the port 24.

FR 2 536 285 A1 discloses a plunger syringe that allows the syringe plunger to be fixed in a specific relative position. Use as a centrifuge syringe is not mentioned. In one embodiment, a syringe plunger having a through-opening is disclosed, with said opening intended to guide a catheter through the syringe plunger.

It is therefore the object of the invention to provide a centrifuge syringe which overcomes the disadvantages of the prior art and, in particular, can be operated easily and continuously.

This object is achieved by a centrifuge syringe comprising a syringe barrel and a syringe plunger axially displaceable in the syringe barrel, whereby a receiving volume enclosed by the syringe barrel and the syringe plunger is formed for a body fluid, preferably blood, wherein the syringe barrel comprises a receiving opening for taking up the body fluid into the receiving volume, and wherein the syringe plunger has a plunger sealing section and a plunger rod undetachably formed on the plunger sealing section, wherein the syringe plunger has a passage extending axially through the plunger sealing section and the plunger rod for a fluid connection between the receiving volume and a removal opening at the end of the plunger rod which faces away from the receiving volume, and wherein the syringe plunger is of such a length that, if the syringe plunger is in a position in the syringe barrel with a minimum receiving volume, the removal opening is arranged essentially at an end of the syringe barrel opposite the receiving volume or protrudes from the syringe barrel, the centrifuge syringe being designed such that a light fraction of the body fluid, after centrifugation preferably plasma, is transferable from the receiving volume into a withdrawal container connected to the removal opening by reducing the receiving volume.

The last-mentioned feature can be implemented, for example, by the passage having a constant diameter (hereinafter referred to as the passage diameter) and/or the passage diameter being at most 4 mm, at most 3 mm or preferably essentially 2 mm at all points.

On the one hand, the syringe plunger of the centrifuge syringe according to the invention is of such a length that the removal opening or, respectively, the syringe plunger can be grasped by the user at any time. On the other hand, the syringe plunger has a passage enabling the withdrawal of body fluid such as plasma from the receiving volume of the centrifuge syringe without the need to break off the syringe plunger at a predetermined breaking point or to remove it from the syringe barrel. The centrifuge syringe according to the invention thus enables simple operation without modular change parts and allows body fluid such as plasma to be withdrawn with particular ease.

Due to the small number of components, the centrifuge syringe according to the invention is furthermore particularly inexpensive to manufacture and less error-prone than prior art centrifuge syringes. However, the centrifuge syringe according to the invention can furthermore be used with existing instruments and centrifuges.

The following preferred embodiments are described for the special case of blood as a body fluid, in which case plasma is withdrawn from the centrifuge syringe via the passage, but with the invention not being limited to the case of being applied to blood or plasma, respectively. In general, the centrifuge syringe according to the invention and the method according to the invention would be applicable to any body fluid and would be advantageous when separation of a light fraction of the body fluid is desired.

The extraction of plasma from (autologous) blood may be desired for various reasons. Plasma or, respectively, preparations comprising plasma are used for a number of medical and/or cosmetic applications. Possible applications of plasma preparations that can be obtained with the centrifuge syringe according to the invention occur, for example, in maxillofacial surgery, traumatology, orthopaedics, aesthetic surgery, dermatology, otorhinolaryngology, ophthalmology, gynaecology, and others. The therapeutic benefit of plasma preparations, especially those derived from autologous blood, is attributed, among other things, to the high content of platelets (hence also known as platelet-rich plasma, PRP) and extracellular vesicles (including exosomes and microvesicles). Another English term for these plasma preparations is also platelet and extracellular vesicle-rich plasma (PVRP).

In a preferred embodiment, the syringe plunger is transparent at least along part of the passage and has a marking in this area for a maximum withdrawal amount. This allows a maximum amount of plasma to be removed from the centrifuge syringe without the risk of accidentally removing red or white blood cells, too, during the withdrawal of plasma. This is made possible by the colour difference between plasma, red blood cells, and white blood cells.

In a further preferred embodiment, the removal opening has a removal connection which is preferably designed as a female Luer connection or a female Luer lock connection. As a result, a withdrawal container, such as, for example, a standard syringe with a male Luer connection or Luer lock connection, can be connected directly to the syringe plunger of the barrel syringe according to the invention. Since no additional elements such as connecting tubes have to be used, the distance between the receiving volume and the withdrawal container or, respectively, the additional syringe is reduced, whereby more plasma can be transferred into the additional syringe or, respectively, into the withdrawal container.

Furthermore, the syringe barrel preferably comprises a receiving opening for taking up the body fluid into the receiving volume, the opening comprising a receiving connection preferably designed as a male Luer connection or a male Luer lock connection. As a result, the centrifuge syringe according to the invention can be used in combination with pre-existing instruments and devices without the need for additional connections.

While the plunger sealing section and the plunger rod are formed so as not to be detachable from each other and, for example, are manufactured as a single piece, the plunger sealing section can furthermore comprise a separate sealing element which seals the plunger sealing section in the circumferential direction in the syringe barrel. Thus, the sealing element is preferably designed as a separate component or is not integrally formed on the syringe plunger, respectively.

In a particularly preferred embodiment, the passage has a constant diameter (passage diameter), i.e., through both the plunger sealing section and the plunger rod. The passage thus has, for example, a cylindrical shape and extends with a constant diameter from the opening of the passage in the syringe plunger on the side of the plunger sealing section up to the opening of the passage on the side of the removal opening of the plunger sealing section. On the side of the removal opening, the passage can merge into the removal connection, where a diameter larger than the passage diameter can be present.

Particularly preferably, it can be envisaged that the passage has a passage diameter of between 2 mm and 4 mm at all points, with the passage diameter preferably being essentially 2 mm. On the one hand, the passage diameter should not be too large so that little air needs to be withdrawn initially and thus only a small residual volume of plasma remains in the passage after removal. At the same time, the passage diameter should not be chosen such that it will be too small, as this could lead to damage to the plasma components due to excessive shearing. A passage diameter of 2 mm has turned out to be appropriate under these requirements.

The passage is in fluid connection with the receiving volume. In one embodiment, further measures can be envisaged, via which the fluid connection is mechanically interrupted, for example, by a needle-shaped plug, also referred to as a mandrel, filling and sealing the volume of the passage. The mandrel can be formed as part of the closure for the removal opening, such as of a thumb press, and is removed together with this closure/the thumb press before the passage as such serves the function of extracting body fluid, preferably plasma.

For stability reasons, the plunger rod preferably has an outer diameter of between 3 mm and 6 mm at all points, with the outer diameter preferably being essentially 4 mm. The above-mentioned marking can be formed by a local elevation in the outer diameter. For further stabilization of the plunger rod, additional measures such as stabilizing ribs can be provided on the plunger rod outside the passage.

In a further aspect, the invention provides a method of using a centrifuge syringe according to the invention for withdrawing body fluid, preferably plasma according to one of the aforementioned embodiments, comprising the following steps:
closing the removal opening using a thumb press;
taking up body fluid into the receiving volume by pulling out the syringe plunger;
closing the receiving opening using a closure stopper;
centrifuging the centrifuge syringe;
removing the thumb press;
connecting a withdrawal container, preferably an additional syringe, to the removal opening;
transferring a lighter fraction of the body fluid, preferably of the plasma, which is present after centrifugation, from the receiving volume into the withdrawal container or, respectively, into the additional syringe by reducing the receiving volume.

This method has the same advantages as the centrifuge syringe explained above and, in particular, enables easy operation of the centrifuge syringe without damaging it and without attaching or installing modular change parts during use.

In a preferred embodiment, when using a centrifuge syringe, the transfer of the body fluid, preferably of the plasma, into the additional syringe occurs by reducing the receiving volume until a colour transition is visible at the marking. By way of this marking, the user can quickly identify when to stop the transfer of plasma into the additional syringe in order to prevent red or white blood cells from entering into the additional syringe.

Moreover, in the aforementioned method of using the centrifuge syringe, it is particularly preferred if the additional syringe is a standard syringe with a male Luer connection or a male Luer lock connection, as, in this case, the additional syringe can be attached directly to the syringe plunger.

Particularly preferably, the withdrawal container or, respectively, the additional syringe can have a capacity that is smaller than the receiving volume of the centrifuge syringe, wherein the steps of connecting and transferring are repeated with multiple withdrawal containers or, respectively, additional syringes until no further lighter fraction of the body fluid, preferably no further plasma, can be removed from the receiving volume. As a result, the plasma in the centrifuge syringe can be distributed to several syringes, for example, several smaller syringes, without the need for an additional instrument.

In general, it is therefore preferred if the maximum receiving volume of the centrifuge syringe is 2 to 20 times larger than the capacity of the additional syringe or, respectively, of the withdrawal containers.

According to an alternative embodiment variant, the maximum receiving volume of the centrifuge syringe essentially corresponds to the capacity of the additional syringe or, respectively, of the withdrawal containers.

Suitable materials and manufacturing methods for the syringe component are known from the prior art. Suitable materials include, for example, polymers based on polypropylene and polyethylene. Surface treatments, for example, with lubricants such as silicone oil or additives such as plasticizers can optimize the properties. An elastomer, particularly a thermoplastic elastomer, can also be considered for the sealing element. When making a choice, a person skilled in the art will consider not only the mechanical properties but also the compatibility with sterilization processes. Examples of sterilization processes are based on the use of gamma rays or ethylene oxide.

In a preferred embodiment, the centrifuge syringe is sterile.

Advantageous and non-limiting embodiments of the invention are explained in further detail below with reference to the drawings.

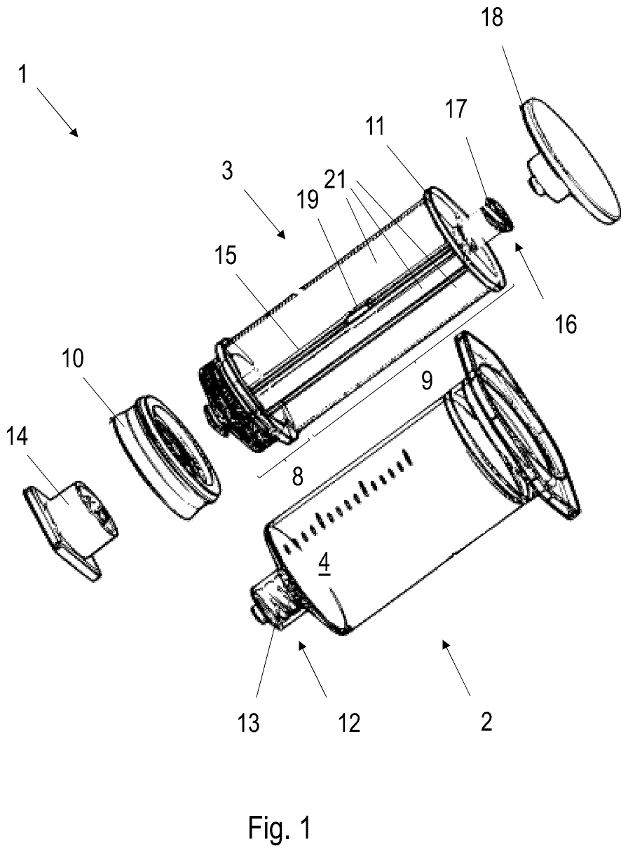

FIG. 1 shows the centrifuge syringe according to the invention in an exploded view.

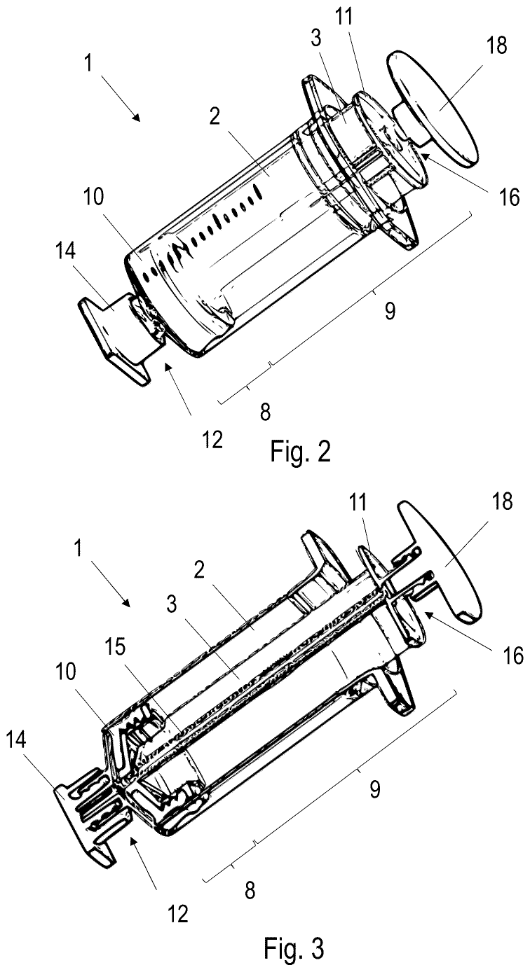

FIG. 2 shows the syringe of FIG. 1 in an assembled state in a perspective view.

FIG. 3 shows the syringe of FIG. 2 in a sectional view.

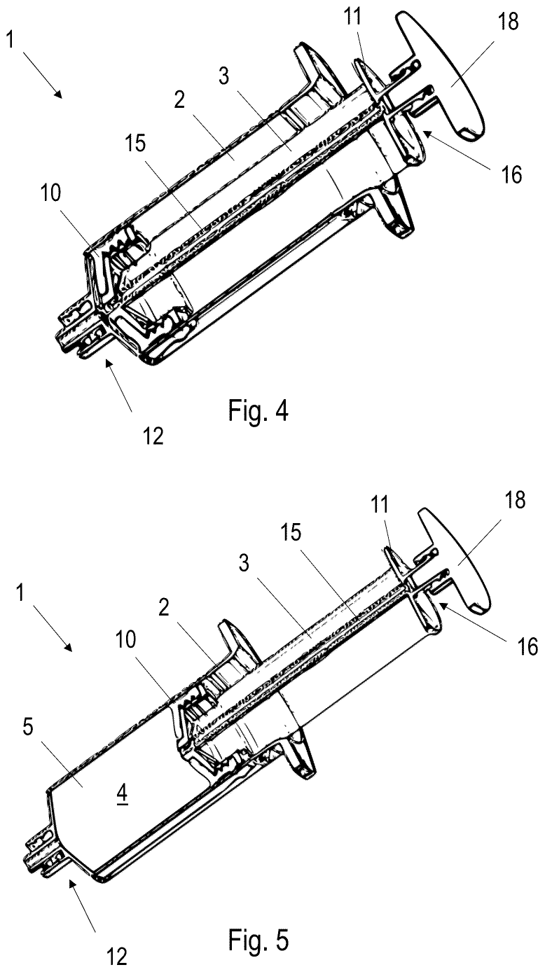

FIG. 4 shows the syringe of FIG. 3 with the closure stopper removed.

FIG. 5 shows the syringe of FIG. 4 after a body fluid has been received.

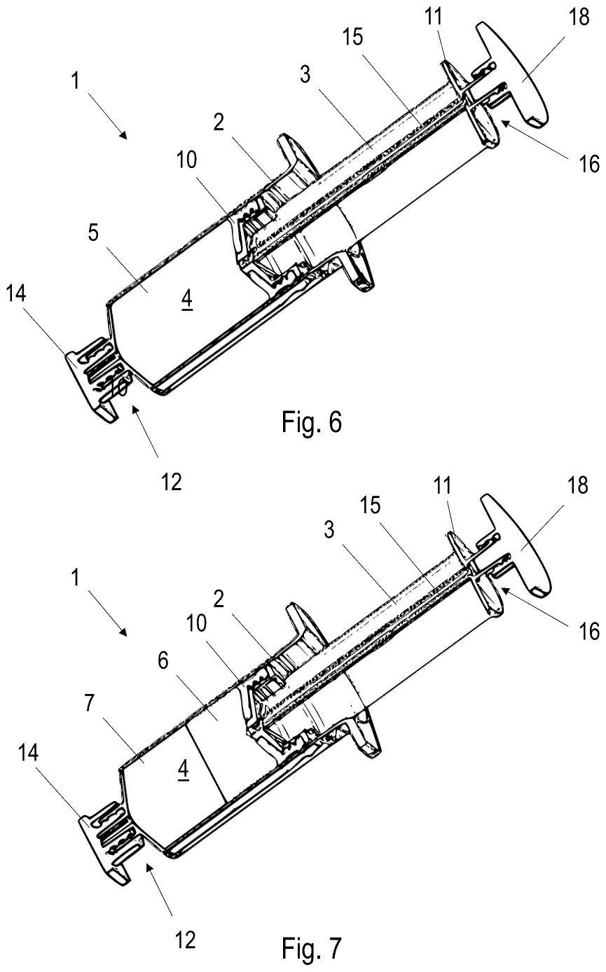

FIG. 6 shows the syringe of FIG. 5 with an attached closure stopper.

FIG. 7 shows the syringe of FIG. 6 after centrifugation.

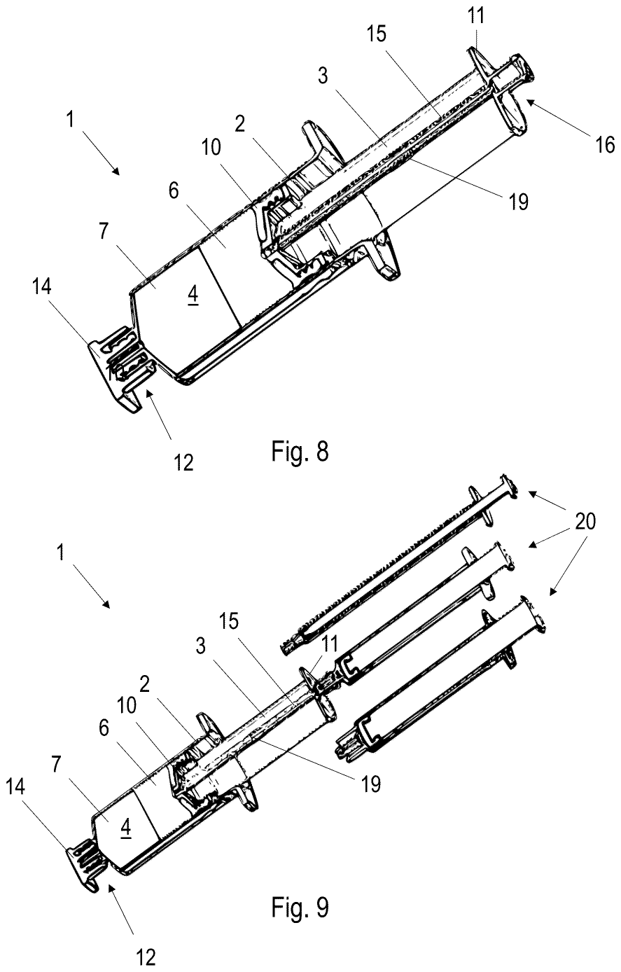

FIG. 8 shows the syringe of FIG. 7 with the thumb press removed on the syringe plunger.

FIGS. 9 to 11 show the extraction of blood plasma from the syringe of FIG. 8 using a standard syringe.

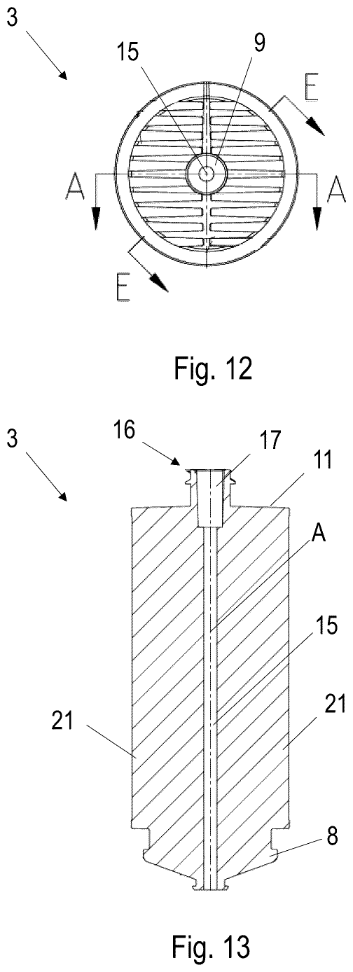

FIGS. 12 to 14 show a preferred embodiment of a syringe plunger.

FIG. 1 shows a centrifuge syringe 1 comprising a syringe barrel 2 and a syringe plunger 3 axially displaceable in the syringe barrel 2. Both the syringe barrel 2 and the syringe plunger 3 are generally made of plastic, in particular transparent plastic, and can be manufactured, for example, by injection moulding methods.

The purpose of the centrifuge syringe 1 described herein is to receive a body fluid such as blood 5, as can be seen in FIG. 5, in a receiving volume 4 of the centrifuge syringe 1 and to subsequently separate it by centrifugation, in the case of blood 5, into plasma 6, red blood cells and white blood cells, as collectively designated by reference numeral 7 in FIGS. 7-11. The centrifuge syringe 1 is described below based on the example of using blood 5, although all embodiments could also be implemented with another body fluid. In this case, the body fluid separates through a centrifugation process into a lighter fraction (in the special case of blood 5, into plasma 6) and a heavier fraction (in the special case of blood 5, into red blood cells and white blood cells 7). All subsequent references to plasma 6 can thus be understood as references to the lighter fraction, and all references to red blood cells and white blood cells 7 can be understood as references to the heavier fraction.

When the syringe plunger 3 is inserted into the syringe barrel 2, a receiving volume 4 is enclosed by these two elements. When the syringe plunger 3 is pushed completely into the syringe barrel 2, as illustrated, for example, in FIGS. 2 to 4, the receiving volume 4 is as small as possible. When the syringe plunger 3 is pulled out of the syringe barrel 2, the receiving volume 4 increases, as can be seen, in particular, in FIGS. 5 to 8.

To enable the syringe plunger 3 to be handled with ease, it comprises a plunger sealing section 8 and a plunger rod 9 undetachably formed on the plunger sealing section 8. For example, the plunger sealing section 8 and the plunger rod 9 can be integrally manufactured as an injection-moulded part. The plunger sealing section 8 is located at the end of the syringe plunger 3 which faces the receiving volume 4. For better sealing, a sealing element 10, such as a sealing ring, can be placed on the plunger sealing section 8, the outer diameter of which essentially corresponds to the inner diameter of the syringe barrel 2.

To prevent the syringe plunger 3 from being pulled out of the syringe barrel 2 in an uncontrolled manner, the syringe barrel 2 can be provided with a lock. The lock can be designed as a circular constriction in the inner wall of the syringe plunger. The constriction is arranged approximately at the height at which the plunger sealing section 8 is located when the syringe plunger 3 is pulled out to its maximum extent and prevents the syringe plunger 3 from slipping completely out of the syringe barrel 2 during normal handling.

To take up blood 5 into the receiving volume 4, the syringe barrel 2 has a receiving opening 12 through which blood can be introduced into the receiving volume 4 from the outside. The receiving opening 12 can have a receiving connection 13 to be connected to compatible devices. The receiving connection 13 can be designed, for example, as a male Luer connection or as a male Luer lock connection. For closing the receiving opening 12, the centrifuge syringe 1 can comprise a closure stopper 14, which is depicted, for example, in FIG. 3 and is preferably designed with a female Luer connection or a female Luer lock connection. The inner diameter of the receiving opening 12 preferably has a width of at least 2 mm. With a receiving opening 12 having an inner diameter of at least 2 mm, mechanical loads and shear forces can be reduced while the body fluid is being received, which is advantageous as they could, for example, promote a blood coagulation process, thus influencing the fractionability of the body fluid. The flow profile also has a positive effect if the inner diameter of the receiving opening 12 is expanded towards the receiving volume 4 as smoothly as possible (approximately to up to 3.6 mm).

For the removal of plasma 6 from the receiving volume 4 after centrifugation, which will be explained in detail below, the syringe plunger 3 is formed with a passage 15 extending axially through the plunger sealing section 8 and the plunger rod 9. The passage 15 extends from the plunger sealing section 8 to receive plasma 6 there, through the plunger rod 9 and ends there in a removal opening 16. Thus, the passage 15 enables a fluid connection between the receiving volume 4 and the removal opening 16, which can be formed, for example, at an upper end 11 of the syringe plunger 3.

Taken together, the plunger sealing section 8 and the plunger rod 9 have a length of such a size that the removal opening 16 protrudes from the syringe barrel 2 when the syringe plunger 3 is in a position in the syringe barrel 2 with a minimum receiving volume 4, as shown in FIGS. 2 to 4. According to the invention, the syringe plunger 3 has such a length that the removal opening 16 is arranged essentially at an end of the syringe barrel 2 opposite the receiving volume 4 or protrudes from the syringe barrel 2 when the syringe plunger 3 is in a position in the syringe barrel 2 with a minimum receiving volume 4. Moreover, for easier handling, the syringe plunger 3 can, for example, have an upper end 11 of the plunger rod 9 that is easy to grip and faces away from the plunger sealing section 8. According to a preferred embodiment, which is illustrated in FIGS. 2, 3 and 4, the upper end 11 that is easy to grip is located outside the syringe barrel 2 when the syringe plunger 3 is positioned in the syringe barrel 2 with a minimum receiving volume 4. For being easy to grip, the upper end 11 can be designed as an end surface which essentially has a diameter that corresponds to the inner diameter of the syringe barrel 2.

The removal opening 16 can, for example, have a removal connection 17, which can be designed as a female Luer connection or as a female Luer lock connection. For closing the passage 15 and preventing an unwanted escape of blood 5 or plasma 6 from the receiving volume 4, the removal opening 16 or, respectively, the removal connection 17 can be closed by a thumb press 18, which is preferably designed with a male Luer connection or a male Luer lock connection. Preferably, the thumb press 18 is arranged completely outside the syringe barrel 2 when it is connected to the removal opening 16 or to the removal connection 17, respectively. In one embodiment, the thumb press 18 forms a mandrel, which extends as a needle-shaped plug into the passage 15 when the removal opening 16 is being closed, completely filling it. Since the mandrel blocks the passage 15, the volume of the passage 15 is prevented from being in fluid connection with the receiving volume 4 during filling and centrifugation.

The passage is preferably designed so as to be transparent so that a user is able to detect whether blood 5, blood plasma 6 and/or white or red blood cells 7 are present in the passage 15. Preferably, the passage 15 can also have a marking 19 indicating a maximum withdrawal amount, as will be described in detail below.

The use of the centrifuge syringe 1 will now be explained in detail below with reference to FIGS. 2 to 11. The centrifuge syringe 1 can be sold, for example, in the state as shown in FIGS. 2 and 3. The centrifuge syringe 1 can preferably be sold in a hard blister pack, in which a closure stopper 14 is enclosed separately, and the hard blister is tightly sealed with a film. In this case, the syringe plunger 3 is inserted into the syringe barrel 2 as far as possible in order to achieve a minimum length for the centrifuge syringe 1. The removal opening 16 of the syringe plunger 3 is closed with a thumb press 18, and the receiving opening 12 of the syringe barrel 2 can be closed with a closure stopper 14. Alternatively, the closure stopper 14 can be added to the centrifuge syringe 1 in a sealed hard blister.

To take up blood 5 into the receiving volume 4, the closure stopper 14 of the receiving opening 12 of the centrifuge syringe 1 is removed, as depicted in FIG. 4. Alternatively, a ready-to-use centrifuge syringe 1 can also be removed, for example, from a hard blister pack, and the receiving opening 12 or, respectively, the receiving connection 13 can be directly or indirectly connected to a blood source. The blood source can be the human body or an artificial container in which blood 5 is located which has already been received previously. During this process, the thumb press 18 continues to close the removal opening 16 of the syringe plunger 3 and, in the version with a mandrel, also the passage 15, if necessary.

To take up blood 5 into the receiving volume 4, the syringe plunger 3 is pulled out as shown in FIG. 5 so that the receiving volume 4 fills with blood 5. With this centrifuge syringe 1, the syringe plunger 3 is pulled out with particular ease, since the removal opening 16 is arranged essentially at an end of the syringe barrel 2 opposite the receiving volume 4 or protrudes from the syringe barrel 2. Preferably, the thumb press 18 connected to the removal opening 16 is gripped as the syringe plunger 3 is being pulled out. Subsequently, the receiving opening 12 or, respectively, the receiving connection 13 is closed with the closure stopper 14 so that the blood 5 cannot escape from the receiving volume 4, see FIG. 6.

Afterwards, the centrifuge syringe 1 can be introduced into a centrifuge, which is not illustrated in any further detail, whereby the blood 5 separates into plasma 6 and red or white blood cells 7, as shown in FIG. 7. Centrifugation is typically performed such that the plasma 6 is located on the side of the receiving volume 4 which faces the syringe plunger 3. The red blood cells are arranged on the side of the receiving volume 4 which faces the receiving opening 12. As a result, it is impossible to directly remove the plasma 6 from the receiving volume 4 through the receiving opening 12.

However, since the syringe plunger 3 of the centrifuge syringe according to the invention is equipped with the passage 15 described above, plasma 6 can be removed through the syringe plunger 3. For this purpose, the thumb press 18 of the removal opening 16 is removed, as shown in FIG. 8, and another syringe 20 is connected to the removal opening 16, see FIG. 9. Alternatively, any suitable withdrawal container can also be connected to the removal opening 16.

The additional syringe 20 can be a conventional syringe or standard syringe. Preferably, the additional syringe 20 has a receiving connection that is compatible with the removal connection 17 of the syringe plunger 3 of the centrifuge syringe 1. For example, the additional syringe 20 can be a standard syringe with a male Luer connection or a male Luer lock connection. As a result, the additional syringe 20 can be connected to the removal connection 17 directly and without further connections.

The capacity of the additional syringe 20 or, respectively, of the withdrawal container can be chosen arbitrarily, but is preferably equal to or smaller than the receiving volume 4 of the centrifuge syringe 1. For example, the maximum receiving volume of the centrifuge syringe is 2 to 20 times, preferably 5 to 10 times, larger than the capacity of the additional syringe 20 so that the plasma 6 located in the receiving volume 4 can be distributed among several additional syringes 20. Depending on the desired application, the receiving volume 4 of the centrifuge syringe 1 can be, for example, 10 ml, 15 ml, 18 ml or 20 ml, although other orders of magnitude could also be selected. The capacity of the additional syringe is, for example, 0.5 ml, 1 ml, 2 ml or 5 ml, although other orders of magnitude could also be selected in this case. In FIG. 9, several additional syringes 20 with different capacities are shown.

According to an alternative embodiment, the maximum receiving volume of the centrifuge syringe 1 essentially corresponds to the capacity of the additional syringe 20 or, respectively, of the withdrawal container(s).

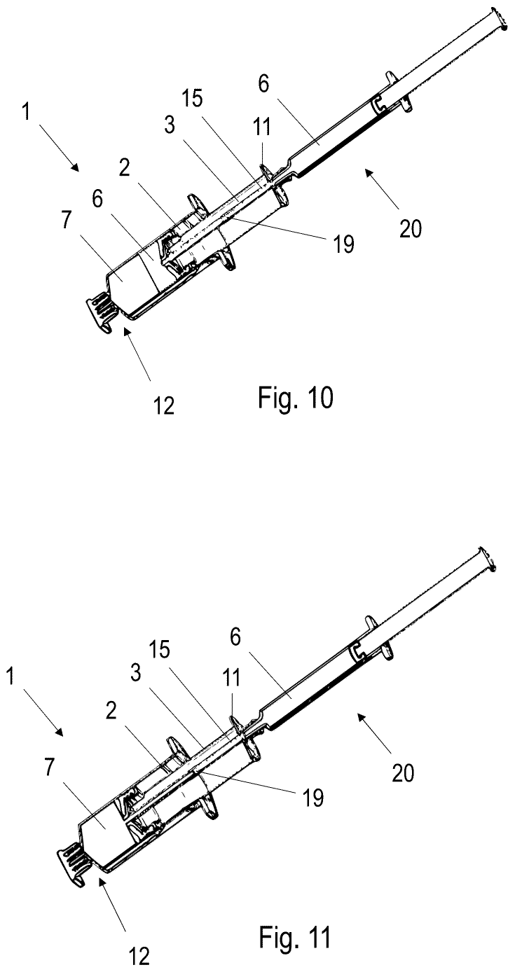

To transfer the plasma 6 from the receiving volume 4 into the additional syringe 20, the receiving volume 4 of the centrifuge syringe 1 is reduced, whereby plasma 6 flows from the receiving volume 4 through the passage 15 into the additional syringe 20, see FIG. 10. The reduction of the receiving volume 4 can be achieved either by pushing the syringe plunger 3 into the syringe barrel 2 manually or in a machine-operated manner, for example, by means of the upper end 11 of the plunger rod 3, or preferably by pulling a syringe barrel out of the additional syringe 20.

As is known per se to a person skilled in the art, plasma 6 can easily be distinguished visually at least from red blood cells 7. If the passage 15 is transparent at least in sections, it is possible to quickly determine during the transfer of plasma 6 into the additional syringe 20 whether plasma 6 is still being transferred into the additional syringe 20 or whether unwanted red or white blood cells are being moved into the additional syringe 20. If the passage 15 has a marking 19 for a maximum withdrawal amount, the user can quickly identify when to interrupt the process of transferring the plasma 6 into the additional syringe 20 to ensure that no white or red blood cells are transferred into the additional syringe 20.

The state in which a colour transition from the plasma 6 to the white or red blood cells 7 is visible at the marking 19 and hence a maximum withdrawal volume has been achieved, is illustrated in FIG. 11.

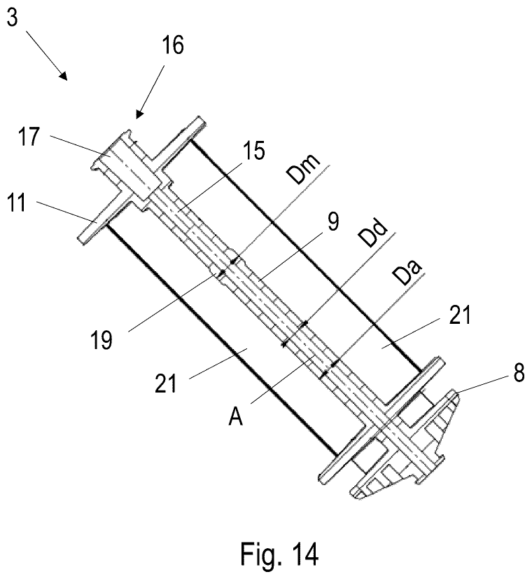

FIGS. 12 to 14 show a preferred embodiment of the syringe plunger 3. As described above, the syringe plunger 3 has a plunger sealing section 8 and a plunger rod 9 undetachably formed on the plunger sealing section 8. FIG. 12 shows the syringe plunger 3 from the direction of the receiving opening 12. FIG. 13 shows the syringe plunger 3 in section A-A of FIG. 12. FIG. 14 shows the syringe plunger 3 in section E-E of FIG. 12.

It can be seen, in particular, from FIGS. 13 and 14 that the passage 15 runs through both the plunger sealing section 8 and the plunger rod 9. The passage 15 preferably runs symmetrically around an axis of rotation or an axis of symmetry A of the syringe plunger 3 or the centrifuge syringe 1, respectively. In preferred embodiments, the passage 15 has a constant passage diameter Dd and optionally ends in an enlarged opening in the area of the removal opening 16 to form the removal connection 17. In other words, in the area of the plunger sealing section 8, the passage 15 has the same passage diameter Dd as in the area of the plunger rod 9. In other embodiments, however, the diameter of the passage could also vary.

In general, it is preferred if the passage 15 has a maximum passage diameter Dd of 4 mm, 3 mm or 2 mm at all points. It is also preferred if the passage has a passage diameter Dd of at least 2 mm or 3 mm at all points. According to the invention, several factors play a role in these considerations. On the one hand, the passage diameter Dd should not be chosen such that it will be too small, as this would lead to damage to the plasma components due to excessive shearing. On the other hand, the passage diameter Dd also should not be chosen such that it will be too large, as more air would have to be withdrawn initially and a larger residual volume of plasma would remain in the passage 15. A passage diameter Dd of 1.9 mm to 2.5 mm, e.g., essentially of 2 mm, has therefore proved to be particularly advantageous. Preferably, the passage diameter Dd is, at most, one-fifth or one-tenth of the inner diameter of the syringe barrel 2.

For example, the plunger rod 9 as such could have an outer diameter Da of 3 mm to 6 mm. With a passage diameter Dd of 2 mm and an outer diameter of 4 mm, the plunger rod 9 would thus have a wall thickness of 1 mm, which involves sufficient stability. In general, the plunger rod 9 can have, for example, a wall thickness of at least 0.5 mm, at least 0.8 mm, at least 1 mm or at least 2 mm, or, respectively, a maximum of 1 mm, a maximum of 3 mm or a maximum of 4 mm. Moreover, the marking 19 explained above can be formed by the plunger rod 9 having a locally increased diameter Dm, which is, for example, at least 0.2 mm, in particular essentially 0.5 mm, larger than the outer diameter Da.

Particularly through the measures explained above, it can be achieved that plasma 6 can be transferred from the receiving volume 4 into a withdrawal container connected to the removal opening 16 by reducing the receiving volume 4, since the passage 15 has just a particularly small volume.

Furthermore, the syringe plunger 3 can have at least one, preferably at least four or exactly four stabilizing ribs 21, which are arranged symmetrically around the axis of symmetry A or the syringe plunger 3, respectively. The stabilizing ribs 21 can extend between the plunger sealing section 8 and the aforementioned upper end 11.

1. A centrifuge syringe (1) comprising a syringe barrel (2) and a syringe plunger (3) axially displaceable in the syringe barrel (2), whereby a receiving volume (4) enclosed by the syringe barrel (2) and the syringe plunger (3) is formed for a body fluid, preferably blood (5),
wherein the syringe barrel (2) comprises a receiving opening (12) for taking up the body fluid into the receiving volume (4),
characterized in that
the syringe plunger (3) has a plunger sealing section (8) and a plunger rod (9) undetachably formed on the plunger sealing section (8), wherein the syringe plunger (3) has a passage (15) extending axially through the plunger sealing section (8) and the plunger rod (9) for a fluid connection between the receiving volume (4) and a removal opening (16) at the end of the plunger rod (9) which faces away from the receiving volume (4), and
wherein the syringe plunger (3) is of such a length that, if the syringe plunger (3) is in a position in the syringe barrel (2) with a minimum receiving volume (4), the removal opening (16) is arranged essentially at an end of the syringe barrel (2) opposite the receiving volume (4) or protrudes from the syringe barrel (2),
the centrifuge syringe (1) being designed such that a body fluid, preferably plasma (6) after centrifugation, can be transferred from the receiving volume (4) into a withdrawal container connected to the removal opening (16) by reducing the receiving volume (4).

2. The centrifuge syringe (1) according to claim 1, wherein the syringe plunger (3) is transparent at least along part of the passage (15) and has a marking (19) in this area for a maximum withdrawal amount.

3. The centrifuge syringe (1) according to claim 1, wherein the removal opening (16) has a removal connection (17) which is preferably designed as a female Luer connection or a female Luer lock connection.

4. The centrifuge syringe (1) according to claim 1, wherein the receiving opening (12) has a receiving connection (13) which is preferably designed as a male Luer connection or a male Luer lock connection.

5. The centrifuge syringe (1) according to claim 1, wherein the plunger sealing section (8) comprises a separate sealing element (10) which seals the plunger sealing section (8) in the circumferential direction in the syringe barrel (2).

6. The centrifuge syringe (1) according to claim 1, wherein the passage (15) has a constant passage diameter (Dd).

7. The centrifuge syringe (1) according to claim 1, wherein the passage (15) has a passage diameter (Dd) of between 2 mm and 4 mm, with the passage diameter (Dd) preferably being essentially 2 mm.

8. centrifuge syringe (1) according to claim 1, wherein the plunger rod (9) has an outer diameter (Da) of between 3 mm and 6 mm, with the outer diameter (Da) preferably being essentially 4 mm.

9. A method of using a centrifuge syringe (1) according to claim 1 for withdrawing body fluid, preferably plasma (6), comprising the following steps:
closing the removal opening (16) using a thumb press (18);
taking up body fluid into the receiving volume (4) by pulling out the syringe plunger (3);
closing the receiving opening (12) using a closure stopper (14);
centrifuging the centrifuge syringe (1);
removing the thumb press (18);
connecting a withdrawal container, preferably an additional syringe (20), to the removal opening (16);
transferring a lighter fraction of the body fluid, preferably of the plasma (6), which is present after centrifugation, from the receiving volume (4) into the withdrawal container or, respectively, into the additional syringe (20) by reducing the receiving volume (4).

10. The method according to claim 9, wherein the syringe plunger (3) is transparent at least along part of the passage (15) and has a marking (19) in this area for a maximum withdrawal amount, wherein the transfer of the body fluid, preferably of the plasma (6), into the withdrawal container or, respectively, into the additional syringe (20) occurs by reducing the receiving volume (4) until a colour transition is visible at the marking (19).

11. The method according to claim 9, wherein the receiving opening (12) has a receiving connection (13) which is preferably designed as a male Luer connection or a male Luer lock connection, wherein the additional syringe (20) is a standard syringe with a male Luer connection or a male Luer lock connection.

12. The method according to claim 9, wherein the withdrawal container or, respectively, the additional syringe (20) has a capacity that is smaller than the maximum receiving volume (4) of the centrifuge syringe (1), wherein the steps of connecting and transferring are repeated with several additional withdrawal containers or, respectively, several additional syringes (20) until no further lighter fraction of the body fluid, preferably no further plasma (6), can be removed from the receiving volume (4).

13. The method according to claim 9, wherein the maximum receiving volume (4) of the centrifuge syringe (1) is 2 to 20 times larger than the capacity of the withdrawal containers or, respectively, of the additional syringes (20).

14. The method according to claim 9, wherein the withdrawal container or, respectively, the additional syringe (20) has a capacity which essentially corresponds to the maximum receiving volume (4) of the centrifuge syringe (1).
